(19) United States
(12) Patent Application Publication
(10) Pub. No.: US 2013/0110173 A1
(43) Pub. Date: May 2, 2013

(54) ATTACHMENT MECHANISM FOR MATERIAL AND BONE

(71) Applicants: Thomas Carls, Memphis, TN (US); Chris Johnson, Germantown, TN (US); Alison Powers, Memphis, TN (US); Stefan Parent, St. Lambert Quebec (CA); Jeremy Rawlinson, Collierville, TN (US)

(72) Inventors: Thomas Carls, Memphis, TN (US); Chris Johnson, Germantown, TN (US); Alison Powers, Memphis, TN (US); Stefan Parent, St. Lambert Quebec (CA); Jeremy Rawlinson, Collierville, TN (US)

(73) Assignee: WARSAW ORTHOPEDIC, INC., Warsaw, IN (US)

(21) Appl. No.: 13/283,868

(22) Filed: Oct. 28, 2011

(51) Int. Cl. A61B 17/88

(52) U.S. Cl. 606/279; 606/300; 606/301

(57) ABSTRACT

A method for anchoring a portion of material to bone is disclosed. One such method comprises inserting the portion of material through a hole in a fastener, and driving the fastener into the bone such that the portion of material becomes anchored to the bone such that driving the fastener farther into the bone increases tension in the material.

The present disclosure is related to commonly owned and co-pending U.S. application Ser. No. ______ (having Attorney Docket No. P40624.USU1), which has a filing date that is the same as the present disclosure, and which is hereby incorporated herein by reference in its entirety.

### FIELD OF INVENTION

The present invention is directed to systems or mechanisms for affixing material to bone.

### BACKGROUND

The present disclosure relates to mechanisms for affixing material to bone, and more particularly, systems for affixing at least a portion of material to a vertebral body.

### SUMMARY OF THE INVENTION

A method for anchoring a portion of material to bone is disclosed. One such method comprises inserting the portion of material through a hole in a fastener, and driving the fastener into the bone such that the portion of material becomes anchored to the bone such that driving the fastener farther into the bone increases tension in the material.

Additional aspects and features of the present disclosure will be apparent from the detailed description and claims as set forth below.

### BRIEF DESCRIPTION OF THE DRAWINGS

FIG. 1 is a schematic, cross-sectional view of two adjacent vertebral bodies with an intervertebral disc situated in its natural location between the two vertebral bodies;

FIG. 2 is a schematic, side view of the vertebral bodies of FIG. 1 as well as a third vertebral body;

FIG. 3 is a schematic, side view of a fastener for attaching material to bone;

FIG. 4 is a schematic, side view of a fastener in cooperation with a portion of material;

FIG. 5 is another schematic, side view of the attachment mechanism of FIG. 4 after the fastener has been moved farther into the bone;

FIG. 6 is a schematic, side view of a fastener for attaching material to bone;

FIG. 6A is a schematic, top view of the fastener of FIG. 6;

FIG. 7 is a schematic, cross-sectional side view of a fastener for attaching material to bone; and

FIG. 7A is a schematic, top view of the fastener of FIG. 7.

### DETAILED DESCRIPTION

For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

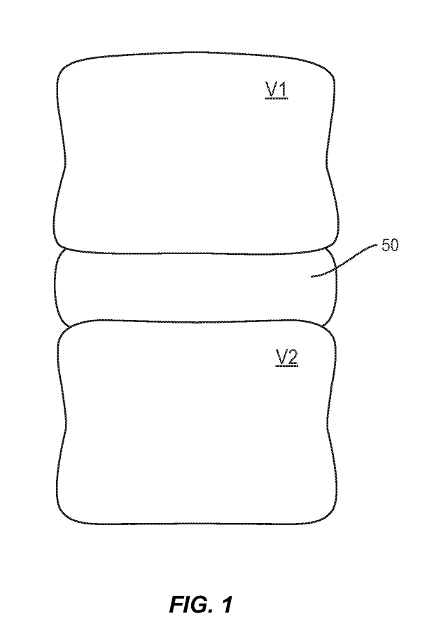

FIG. 1 shows a schematic, cross-sectional view of two adjacent vertebral bodies V1 and V2 with an intervertebral disc 50 situated in its natural location between the two vertebral bodies V1 and V2. As shown in FIG. 1, vertebral body V1 represents a superior vertebral body and V2 represents an inferior vertebral body.

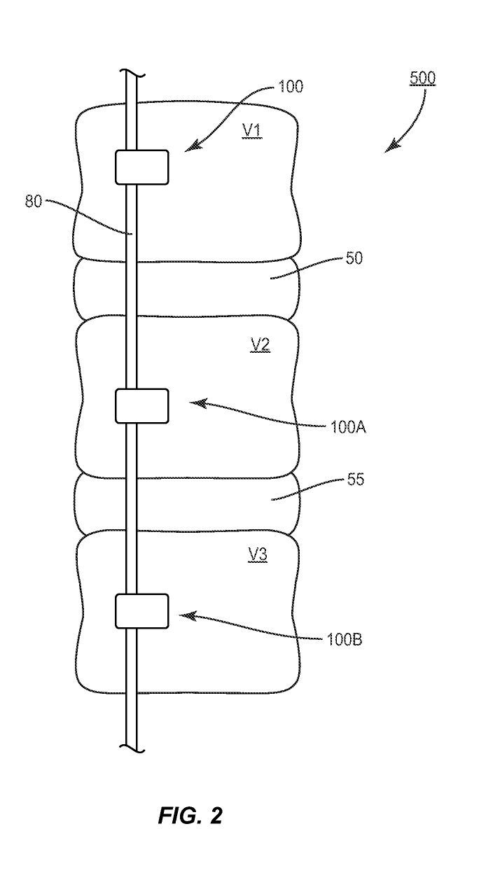

FIG. 2 shows a schematic, side view of the vertebral bodies V1 and V2 of FIG. 1 as well as a third vertebral body V3. As shown in FIG. 2, disc 50 is situated between vertebral bodies V1 and V2, and disc 55 is situated between vertebral bodies V2 and V3. Also shown in FIG. 2 is an anchoring system 500 comprising three anchoring mechanisms, each of which is used to anchor a portion of material 80 to a vertebral body. As show in FIG. 2, there is an anchoring mechanism 100 affixed to vertebral body V1, an anchoring mechanism 100A affixed to vertebral body V2, and an anchoring mechanism 100B affixed to vertebral body V3.

The anchoring mechanisms 100, 100A and/or 100B may be affixed to a variety of locations on the vertebral bodies V1 and V2, for example, they may be affixed to the lateral side surfaces or affixed to the pedicles (not shown) on the posterior section of the vertebral bodies V1, V2 and V3. When affixed to the lateral side surfaces, such an anchoring system 500 may be used to correct certain spinal conditions or deformities in the coronal plane, for example, for use in fusionless spinal treatments to address conditions such as scoliosis. When used in such a manner to treat scoliosis, the anchoring system 500 may be placed on the convex side of the curvature of the spine. Further, when used to treat scoliosis, the anchoring mechanisms 100A, 100B and 100C may be placed on lateral side surfaces of the respective vertebral bodies.

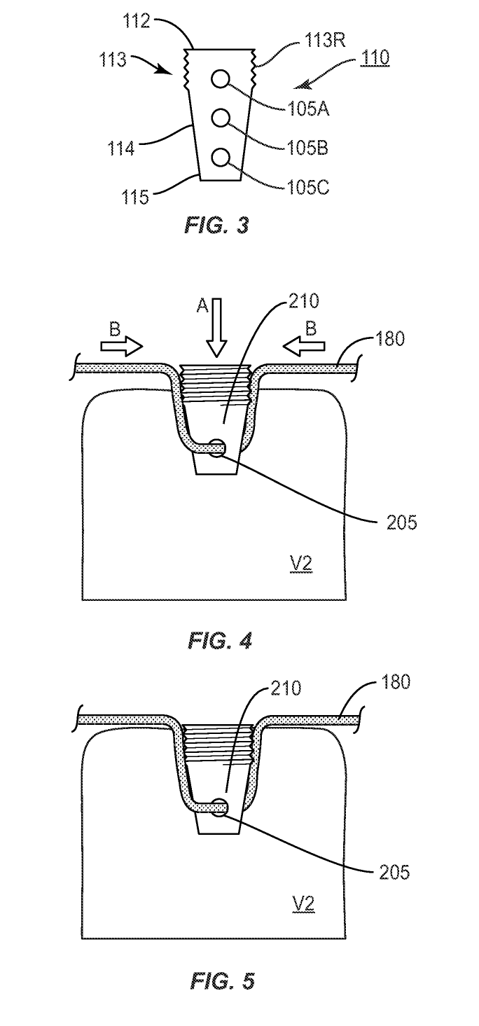

FIG. 3 shows a schematic, side view of a fastener 110 for attaching material to bone. As such, the fastener 110 may serve as an attachment mechanism described above. As shown in FIG. 3, fastener 110 comprises a head 112 located at a proximal portion of the fastener 110, a neck 113, a shank 114 and a distal portion 115. As shown, fastener 110 comprises three holes 105A, 105B and 105C. Hole 105A is located in the neck of the fastener 110, hole 105B is located substantially in the middle of shank 114 of the fastener 110, and hole 105C is located in the distal portion 115 of the fastener 110. In addition, as shown in FIG. 3, the fastener 110 further comprises ridges 113R situated around the neck 113 of the fastener 110. As shown, the ridges 113R may be used to promote affixation of the fastener 110 into bone such as vertebral bodies.

The term “substantially” (or “substantial”) as used herein may be applied to modify any quantitative representation which could permissibly vary without resulting in a change in the basic function to which it is related. For example, while hole 105B may be located substantially in the middle of shank 114, hole 105B need not be located in the exact middle of the shank 114 to serve its desired purpose.

FIG. 4 shows a schematic, side view of a fastener 210 in cooperation with a portion of material 180. A method for anchoring the portion of material 180 to bone V2 is disclosed. The method comprises inserting the portion of material 180 through a hole 205 in the fastener 210, and then driving the fastener 210 into the bone V2 such that the portion of material 180 becomes anchored to the bone V2 such that driving the fastener 210 farther into the bone increases tension in the portion of material 180. Specifically, when a force is applied in the direction “A” and the fastener 210 moves farther into the bone V2 or farther downward in the bone V2, tension in the portion of material 180 increases as the portion of material 180 is pulled in the direction “B.” FIG. 5 shows the attachment mechanism (fastener 210) of FIG. 4 after the fastener 210 has been moved farther into the bone V2. Thus, after inserting the portion of material 180 through the hole 205, one may drive the fastener 210 far enough into the bone V2 to achieve desired tension in the portion of material 180.

With the attachment mechanisms and methods described herein, the portion of material 180 need only be inserted through the hole 205, but other means of affixing the portion of material 180 to the fastener 210 may be employed. For example, the portion of material 180 may be affixed to the fastener 210 by any means know in the art, for example, by gluing, tying a knot or knots, suturing material 180 onto itself, or by effecting a phase change of one or more materials to obtain rigid or solid fusion of such materials. The term “affix” is used herein in a relatively broad sense. That is, the word “affix” is intended to mean verbs such as join, secure, and hold. Accordingly, while the word “affix” may encompass the verb fuse, as in fusing together two items in a rigid manner, it is not limited to such a narrow definition.

As shown in FIGS. 4 and 5, bone V2 represents a vertebral body. As shown, the portion of material 180 may extend from fastener 210 on vertebral body V2 to similar fasteners, for example, on vertebral bodies V1 and V3, as shown in the attachment system 500 of FIG. 2. Another method of increasing tension in a portion of material 180 extending between two bones or between two vertebral bodies is to insert the portion of material 180 through a hole in a distal portion of the fastener 210 rather than through a hole at a more proximal location of the fastener 210. That is, with the system and method of attaching as disclosed herein, inserting the portion of material 80 or 180 through a more proximal hole than through a more distal hole results in an overall increase in tension of the portion of material 80 or 180. Using the fastener 110 of FIG. 3, inserting the portion of material 80 or 180 through hole 105C results in an increase in tension of the portion of material 80 or 180 than if hole 105B or hole 105A was used. Similarly, inserting the portion of material 80 or 180 through hole 105B results in an increase in tension of the portion of material 80 or 180 than if hole 105A was used. Thus, after selecting the hole (for example, 105A, 105B or 105C) that is positioned to achieve the desired tension in the portion of material 80 or 180, one may drive the fastener into the bone. Accordingly, one may achieve desired tension in the portion of material 80 or 180 by manipulating either the location of the hole in a fastener or the distance into the bone that the fastener is driven, or any combination of the two.

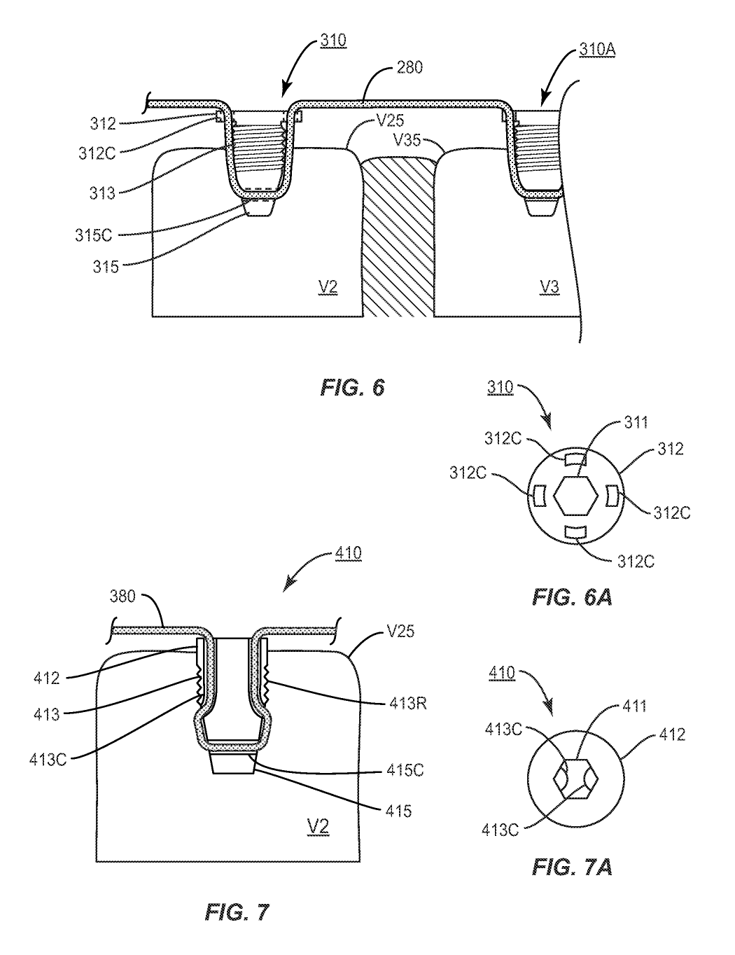

FIG. 6 shows a schematic, side view of a fastener 310 for attaching material to bone. As such, the fastener 310 may serve as an attachment mechanism described above. As shown in FIG. 6, fastener 310 is situated in vertebral body V2 and a fastener 310A (which is similar to that of fastener 310) is situated in vertebral body V3, and the two fasteners 310 and 310A and thereby their respective vertebral bodies V2 and V3 are attached by means of a portion of material 280.

As shown in FIG. 6, fastener 310 comprises a head 312 located at a proximal portion of the fastener 310, a neck 313, ridges 313R situated around the neck 313, and a distal portion 315. As shown, fastener 310 comprises a hole 315C through the distal portion 315, and channels 312C through the head 312. As shown, hole 315C may be used, for example, in a manner similar to that of hole 105C in allowing the portion of material 280 to pass through the fastener 310. Channels 312C allow the portion of material 280 to pass through the head 312 of the fastener 310. In this way, channels 312C allow for the portion of material 280 to avoid contact with any outer surface of a vertebral body, for example, outer surfaces V2S or V3S of vertebral bodies V2 or V3, respectively. Accordingly, as shown in FIG. 6, the portion of material 280 may pass from fastener 310 in vertebral body V2 to fastener 310A in vertebral body V3 without contacting the outer surface of either vertebral body.

FIG. 6A shows a schematic, top view of fastener 310. As shown in FIG. 6A, the head 312 of fastener 310 comprises a tool-engaging recess 311 and channels 312C, which are spaced symmetrically around the perimeter of the head 312. As shown in FIG. 6A, the head 312 comprises four channels 312, but this number (as well as their size) may vary and they need not be spaced symmetrically, as only two channels or pathways are necessary: one channel to guide one end of the portion of material 280 and another channel to guide the other end of the portion of material 280.

FIG. 7 shows a schematic, cross-sectional side view of a fastener 410 for attaching material to bone. As such, the fastener 410 may serve as an attachment mechanism described above. As shown in FIG. 7, fastener 410 is situated in vertebral body V2 and a portion of material 380 may extend from fastener 410 in vertebral body V2 to other fasteners in other portions of bone, for example, in vertebral bodies V1 and V3 as shown in attachment system 500 of FIG. 2.

As shown in FIG. 7, fastener 410 comprises a head 412 located at a proximal portion of the fastener 410, a neck 413, ridges 413R situated around the neck 413, and a distal portion 415. As shown, fastener 410 comprises a hole 415C through the distal portion 415, and channels 413C through the neck 413. As shown, hole 415C may be used, for example, in a manner similar to that of hole 105C in allowing the portion of material 380 to pass through the fastener 410. Channels 413C allow the portion of material 380 to pass through the neck 413 and head 412 of the fastener 410. In this way, channels 413C allow for the portion of material 380 to avoid contact with any outer surface of a vertebral body, for example, outer surface V2S vertebral body V2. Accordingly, as shown in FIG. 7, the portion of material 380 may pass from fastener 410 in vertebral body V2 to, for example, another fastener in another vertebral body without contacting the outer surface of either vertebral body. Note that although the channels 413C are shown situated in the neck 413 of fastener 410, the channels 413C may be situated anywhere between hole 415C and the outer surface V2S.

FIG. 7A shows a schematic, top view of fastener 410. As shown in FIG. 7A, the head 412 of fastener 410 comprises a tool-engaging recess 411 and channels 413C. As shown in FIG. 7A, the head 412 comprises two channels 312, but this number (as well as their size) may vary, as only two channels or pathways are necessary: one channel to guide one end of the portion of material 380 and another channel to guide the other end of the portion of material 380.

With the attachment mechanisms and methods described herein, although the attachment mechanisms (or fastener 110 or 210) are shown as dowels, the fasteners 110 or 210 also could be screws. Further, the dowels (or screws) may comprise synthetic material (for example, various metals or polymers), biologic material (for example, an allograft such as bone) or any combination thereof. In addition, such dowels may be made of a combination comprised of both a synthetic material and a biologic material, or a biologic material that may be considered more than minimally processed.

In the attachment systems and methods described herein, the portions of material are non-rigid and flexible. In addition, the portions of material may be a tether or part of a tether. Further, each portion of material may be a ligament, i.e., synthetic ligament or natural ligament. In addition, each portion of material may be a graft such as an autograft, allograft or xenograft.

Further, the portions of material may be any one or combination of a cloth, metal, solid polymer, fabric, mesh, or other biocompatible material. Some polymer materials may include but not be limited to, any one or combination of polyethylene, polyester, polyvinyl, polyvinyl alcohol, polyacrylonitrile, polyamide, polytetrafluoroethylene, poly-paraphenylene and terephthalamide. In addition, the portions of material may be made of a suture wire of polyester or polyethylene. Further, the portions of material may be elastic, woven, knitted, braided or flexible. Some woven, knitted or braided materials may, for example, include nylon, Dacron®, and/or woven fibers or filaments of polyester, polyethelene, polypropylene, polyetheretherketone (“PEEK”), polytetrafluoroethylene (“PTFE”), and/or woven PEEK. Some elastic materials may, for example, include latex, rubber, silicone, polyurethane, silicone-polyurethane copolymers, and/or polyolefin rubbers. Other suitable materials may, for example, include Gore-Tex®, Kevlar®, Spectra, polyether, polycarbonate urethane, shape memory material with pseudo elastic or superelastic characteristics, metals, metal alloys, and polymers, braided polymers, synthetic resorbable materials such as polyactide, polygycolide, polyorthoester, calcium phosphate, and/or glass, nonresorbable polyethylene, cellulose, materials that are potentially absorbable, and/or materials that are used in making synthetic ligaments. Further, suitable materials may be biodegradable or non-biodegradable. Similarly, suitable materials may be resorbable or non-resorbable. In addition to woven, braided, or knitted structures, the portions of material also may be composed of non-woven structures such as non-woven mesh or chained structures.

All adjustments and alternatives described above are intended to be included within the scope of the invention, as defined exclusively in the following claims. Those skilled in the art also should realize that such modifications and equivalent constructions or methods do not depart from the spirit and scope of the present disclosure, and that they may make various changes, substitutions, and alterations herein without departing from the spirit and scope of the present disclosure. For example, although the attachment systems above are described as being configured to affix a portion of material to a vertebral body, they also may affix a portion of material to any bone. Similarly, although the attachment systems are described as being configured to affix to a pedicle of a vertebral body, they also may affix to other surfaces of a vertebral body such as an anterior, antero-lateral, or lateral face of a vertebral body or any plurality or combination of such surfaces.

Furthermore, as used herein, the terms components and elements may be interchanged. It is understood that all spatial references, such as “superior,” “inferior,” “anterior,” “posterior,” “above,” “lower,” “outside,” “inside,” “higher,” “lower,” “outer,” “inner,” “extended,” “reduced,” “shorter,” “longer,” and “perimeter” are for illustrative purposes and can be varied within the scope of the disclosure.

1. A method for anchoring a portion of material to bone, the method comprising:
inserting the portion of material through a hole in a fastener; and
driving the fastener into the bone such that the portion of material becomes anchored to the bone such that driving the fastener farther into the bone increases tension in the material.

2. The method of claim 1, wherein the fastener is a dowel.

3. The method of claim 3, wherein the step of inserting is performed before the step of driving.

4. The method of claim 4, wherein during the step of inserting, the portion of material is inserted through a hole in a distal portion of the fastener such that the tension in the material is further increased than if the portion of material was inserted through a hole at a more proximal location.

5. The method of claim 1, wherein the hole is located substantially in the middle of the shank of the fastener.

6. The method of claim 1, wherein the hole is located in the neck of the fastener.

7. The method of claim 1, wherein the hole is in the distal portion of the fastener.

8. The method of claim 1, wherein the fastener is a screw.

9. The method of claim 1, wherein the portion of material is flexible.

10. The method of claim 9, wherein the portion of material is part of a tether.

11. The method of claim 1, wherein the portion of material is a ligament.

12. The method of claim 11, wherein the portion of material is a synthetic ligament.

13. The method of claim 1, wherein the portion of material is an allograft.

14. The method of claim 1, wherein the dowel is comprised of synthetic material.

15. The method of claim 1, wherein the dowel is comprised of biological material.

16. The method of claim 1, wherein the dowel is an allograft.

17. The method of claim 2, wherein the dowel comprises ridges in a neck portion of the dowel.

18. A method for anchoring a portion of material to bone, the method comprising:
inserting the portion of material through a hole in a dowel; and
driving the dowel far enough into the bone to achieve desired tension in the material.

19. The method of claim 18, wherein the method is used to treat scoliosis in a fusionless manner.

20. A method for anchoring a portion of material to bone, the method comprising:
inserting the portion of material through a hole in a dowel, the hole being positioned to achieve desired tension in the material; and
driving the dowel into the bone.
